SEQUENCE LISTING (5 sequences)

SEQ ID NO: 1 - DNA, 2048 bp, synthetic construct
  source 1..2048: mol_type = other DNA
atgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatga

SEQ ID NO: 2 - DNA, 20 bp, synthetic construct
  source 1..20: mol_type = other DNA
cgagatccctccaaaatcaa

SEQ ID NO: 3 - DNA, 20 bp, synthetic construct
  source 1..20: mol_type = other DNA
atccacagtcttctgggtgg

SEQ ID NO: 4 - DNA, 20 bp, synthetic construct
  source 1..20: mol_type = other DNA
tcttgctgtggactggattg

SEQ ID NO: 5 - DNA, 20 bp, synthetic construct
  source 1..20: mol_type = other DNA
agcaattcctcgtggttgag

(19) United States
(12) Patent Application Publication
(10) Pub. No.: US 2025/0114481 A1
(43) Pub. Date: Apr. 10, 2025

(54) LENTIVIRAL VECTOR FOR OVEREXPRESSING EGF, RECOMBINANT STEM CELL, AND USE THEREOF

(71) Applicant: Centre for Regenerative Medicine and Health, Hong Kong Institute of Science & Innovation, Chinese, Pak Shek Kok (HK)

(72) Inventors: Dajiang QIN, Pak Shek Kok (HK); Hongjie XU, Pak Shek Kok (HK); Di WU, Pak Shek Kok (HK); Yixia LING, Pak Shek Kok (HK); Xia WANG, Pak Shek Kok (HK)

(21) Appl. No.: 18/927,156

(22) Filed: Oct. 25, 2024

(30) Foreign Application Priority Data
Oct. 26, 2023 (CN) 202311408438.9

(51) Int. Cl. A61K 48/00; A61K 35/28; A61K 38/18; C12N 5/0775; C12N 7/00; C12N 15/86

(52) U.S. Cl. CPC A61K 48/005; A61K 35/28; A61K 38/1808; C12N 5/0665; C12N 7/00; C12N 15/86; C12N 2740/15043; C12N 2830/48

(57) ABSTRACT

The disclosure provides a lentiviral vector for overexpressing Epidermal Growth Factor (EGF), a recombinant stem cell containing the lentiviral vector, and use of the lentiviral vector and the recombinant stem cell containing the lentiviral vector in the preparation of a medicament for the treatment of related diseases. The lentiviral vector of the disclosure comprises a vector plasmid, wherein the vector plasmid comprises a 5′LTR containing a ψ sequence, a 3′LTR, a target gene sequence between the 5′LTR and the 3′LTR, and a promoter sequence and a translation initiation sequence operably linked to the target gene sequence, and the target gene sequence is a nucleotide sequence encoding EGF.

### CROSS-REFERENCE TO RELATED APPLICATIONS

This application claims the priority of CN patent application No. 2023114084389, filed Oct. 26, 2023, the content of which is incorporated herein by reference in its entirety.

### SEQUENCE LISTING

The instant application contains a Sequence Listing which has been submitted in an XML file with the USPTO and is hereby incorporated by reference in its entirety. The Sequence Listing was created on Oct. 10, 2024, is named “SequenceListing.xml”, and is 7,343 bytes in size.

### TECHNICAL FIELD

The disclosure belongs to the field of biotechnology, and specifically relates to a lentiviral vector for overexpressing EGF and a recombinant stem cell containing the lentiviral vector, as well as a preparation method and use thereof.

### BACKGROUND ART

Epidermal growth factor (EGF) plays an important role in the growth, proliferation, and differentiation of various types of cells (especially fibroblasts and keratinocytes). It is an important epidermal growth factor that can stimulate the proliferation of epidermal cells and can be applied to the study of wound healing. Epidermal growth factor was first isolated from the submandibular gland of male mice, and widely exists in different tissues and body fluids of human body. It can stimulate the proliferation and migration of various cells and is used in repairing and healing wounds caused by such as corneal injuries, burns, scalds, and surgeries, and can prevent scar hyperplasia. At present, EGF is used in China as a medical topical preparation. As an active ingredient, it can promote wound repair and guarantee the repair of the epidermis, and it is suitable for the treatment of skin burn or scald wounds (superficial II-degree to deep II-degree of burn or scald wounds), residual wounds, wounds in skin donor area, chronic ulcer wounds and the like.

Mesenchymal stem cells (MSCs) are a class of mesenchymal cells with the potential for multi-lineage differentiation. They are a cell type suitable for immunomodulation and regenerative repair because of their wide range of sources, low immunogenicity, and the creation of a favorable microenvironment for tissue regeneration. It has been elucidated that mesenchymal stem cells transduced with the EGF gene (EGF-MSC) contribute to wound healing. CN113088496A discloses an EGF MSC exosome, which infects MSCs with a lentiviral vector. However, the expression level of EGF slightly increases after editing, and the expression level in the cell supernatant after editing is less than 20 pg/mL, and the concentration of EGF is still very low which was only of the order of pg/mL. Therefore, there is a need to develop a method of increasing the amounts of the expression and secretion of EGF protein.

### SUMMARY OF THE INVENTION

In order to overcome the problem of low levels of the expression and secretion of EGF protein in mesenchymal stem cells, the disclosure provides a lentiviral vector capable of overexpressing EGF and a mesenchymal stem cell containing the lentiviral vector, which can be used in the treatment of wound surfaces repair and healing, tissue repair, and wound healing, or in the prevention of scar hyperplasia.

In order to achieve the objects of the disclosure, in a first aspect, the disclosure provides a lentiviral vector comprising a vector plasmid, wherein the vector plasmid comprises a 5′LTR containing a ψ sequence, a 3′LTR, a target gene sequence between the 5′LTR and the 3′LTR, and a promoter sequence and a translation initiation sequence operably linked to the target gene sequence, and the target gene sequence is a nucleotide sequence encoding EGF.

In some embodiments, the nucleotide sequence encoding EGF is a nucleotide sequence shown in SEQ ID NO: 1 or a nucleotide sequence having at least 85%, at least 88%, at least 90%, at least 92%, at least 95%, at least 97%, at least 98%, at least 99%, or 100% sequence identity to SEQ ID NO: 1.

In some embodiments, the 3′LTR and the 5′LTR comprise one or more modifications.

In some embodiments, the U3 of the 5′LTR and the 3′LTR may be deleted or mutated.

In some embodiments, the 3′LTR is a 3′LTR in which a U3 region is deleted (3′LTR-ΔU3).

In some embodiments, the 5′LTR is a 5′LTR in which a U3 region is deleted (5′LTR-ΔU3).

In some embodiments, the promoter of the 5′LTR is selected from the group consisting of a cytomegalovirus CMV promoter, a Rous sarcoma virus RSV promoter, and a simian virus SV40 promoter, preferably the Rous sarcoma virus RSV promoter.

In some embodiments, the promoter operably linked to the nucleotide encoding EGF is selected from the group consisting of a short elongation factor 1A (EF1α) promoter or a transcriptionally active fragment thereof, an RSV promoter, and a simian virus SV40 promoter, preferably a short elongation factor 1A (EF1α) promoter.

In some embodiments, the nucleotide encoding EGF is operably linked to an EF1α promoter and a Kozak translation initiation sequence.

In some embodiments, the vector plasmid further comprises a nucleotide encoding a screening marker.

In some embodiments, the screening marker is selected from one or more of Luciferase, fluorescent protein, streptavidin binding peptide, puromycin resistance marker, ampicillin resistance marker, kanamycin resistance marker, and neomycin resistance marker. Preferably, the screening marker is an enhanced green fluorescent protein (EGFP).

In some embodiments, the vector plasmid further comprises an SV40 early pA.

In some embodiments, a post-transcriptional regulatory element of the vector plasmid comprises a woodchuck hepatitis virus post-transcriptional regulatory element (WPRE).

In some embodiments, the vector plasmid comprises a retroviral export element. “Retroviral export element” means a cis-acting post-transcriptional regulatory element that regulates the transport of RNA transcripts from the nucleus to the cytoplasm. Preferably, the retroviral export element includes, but is not limited to, a human immunodeficiency virus (HIV) rev response element (RRE) and a hepatitis B virus post-transcriptional regulatory element (HPRE).

In some embodiments, the vector plasmid further comprises a central polypurine region (cPPT) or a central termination sequence (CTS).

Preferably, the cPPT sequence is a cPPT of HIV1.

Preferably, the CTS sequence is a CTS of HIV1.

In some embodiments, the vector plasmid, sequentially from the 5′LTR region to the 3′LTR region, comprises: an RSV promoter, a 5′LTR-ΔU3, a ψ sequence, an RRE, a cPPT, an EF1α promoter, a Kozak translation initiation sequence, a nucleotide encoding EGF, an EGFP, a WRPE, a 3′LTR-ΔU3, and an SV40 early pA.

The lentiviral vector carrying a nucleotide encoding EGF constructed in the disclosure can highly express a promoter and a translation initiation sequence in stem cells. Moreover, since the lentiviral load capacity is limited, and the above elements together with the long EGF sequence are essentially fully loaded, the inventors boldly discarded the antibiotic screening sequence and retained only eGFP as a label for observing and identifying overexpression efficiency. In addition, considering that the EGF gene sequence is already long, no additional signal peptide sequence is added before the EGF gene, and no additional sequences of linker region and transmembrane region are added thereafter. This design can improve the infection efficiency of the lentiviral vector, further improve the expression rate and the accuracy of expression of EGF in infected recombinant stem cells, and furthermore increase the level of protein secretion.

In a second aspect, the disclosure provides a recombinant stem cell, wherein the recombinant stem cell comprises the lentiviral vector of the first aspect and is capable of expressing the target nucleotide carried in the lentiviral vector of the first aspect.

In some embodiments, the recombinant stem cell is a mesenchymal stem cell.

In some embodiments, the mesenchymal stem cell is a human umbilical cord mesenchymal stem cell.

The recombinant stem cell, in particular the mesenchymal stem cell, transfected with the lentiviral vector carrying EGF-coding nucleotides can overexpress the EGF gene. The mesenchymal stem cell is a mesenchymal stem cell having an ability to highly express EGF.

In a third aspect, the disclosure provides a method for the preparation of the recombinant stem cell of the second aspect, wherein the method comprises the following steps:
constructing the lentiviral vector of the first aspect;
transfecting a stem cell with the lentiviral vector constructed in step (1) to obtain the recombinant stem cell of the second aspect; and
culturing the recombinant stem cell obtained in step (2).

In some embodiments, the cell is a stem cell.

In some embodiments, the stem cell is a mesenchymal stem cell. Preferably, the mesenchymal stem cell is a human umbilical cord mesenchymal stem cell.

In some embodiments, the stem cell is a human umbilical cord mesenchymal stem cell transfected with the lentiviral vector carrying the nucleotide encoding EGF.

In some embodiments, in step (2), the seeding density of the stem cells is 1.0×10⁵ to 2.0×10⁵, preferably 1.0×10⁵.

In some embodiments, in step (2), the multiplicity of infection MOI of a virus is 10 to 30, preferably 20 to 30.

In some embodiments, in step (2), the auxiliary transfection reagent used for transfection is selected from Polybrene. Preferably, a working concentration of Polybrene is 5 to 8 μg/mL.

According to the preparation method of the disclosure, overexpressing the EGF gene by lentiviral means significantly increases the levels of expression and secretion of EGF in the recombinant stem cell.

In a fourth aspect, the disclosure provides a pharmaceutical composition, comprising the lentiviral vector of the first aspect, the recombinant stem cell of the second aspect, or the recombinant stem cell obtained by the preparation method of the third aspect, and a pharmaceutically acceptable excipient or carrier.

In a fifth aspect, the disclosure provides use of the lentiviral vector of the first aspect, the recombinant stem cell of the second aspect, the recombinant stem cell obtained by the preparation method of the third aspect, or the pharmaceutical composition of the fourth aspect in the preparation of a medicament for the treatment of wound surfaces repair and healing, tissue repair, and wound healing, or for the prevention of scar hyperplasia.

In some embodiments, the wound surfaces include, but are not limited to, wound surfaces caused by corneal injury, burns, scalds, surgeries, or chronic ulcers, and the like.

Compared with the prior art, the disclosure has at least one of the following beneficial effects:

The lentiviral vector for overexpressing EGF involved in the disclosure is designed to be simple and efficient, with high infection efficiency.

The mesenchymal stem cell infected with an EGF-overexpressing lentivirus involved in the disclosure has an increased EGF at the mRNA level, which can significantly increase the expression of EGF.

The mesenchymal stem cell infected with an EGF-overexpressing lentivirus involved in the disclosure has a significantly increased level of EGF protein secretion in the supernatant.

### BRIEF DESCRIPTION OF THE DRAWINGS

FIG. 1 shows a mapping of the lentiviral vector overexpressing EGF.

FIG. 2 shows the effects of EGF overexpression identified by fluorescence microscopy (A) and flow cytometry (B).

FIG. 3 shows the results of detecting EGF gene transcription levels in MSCs by qPCR method, **** P≤0.0001, compared with the MSC group; ####P≤0.0001, compared with the MSC-EGF M20 group.

FIG. 4 shows the results of detecting the ability of MSCs to secrete EGF by Elisa method, ****P≤0.0001, compared with the MSC group; ####P≤0.0001, compared to the MSC-EGF M20 group.

### DETAILED EMBODIMENTS OF THE INVENTION

The following descriptions are the preferred embodiments of the disclosure, and the scope of the disclosure is not limited to the following preferred embodiments. It should be noted that various changes and modifications made by those skilled in the art based on the concept of the invention all fall within the scope of protection of the disclosure. The reagents used without the manufacturer's indication, are conventional products that can be obtained commercially.

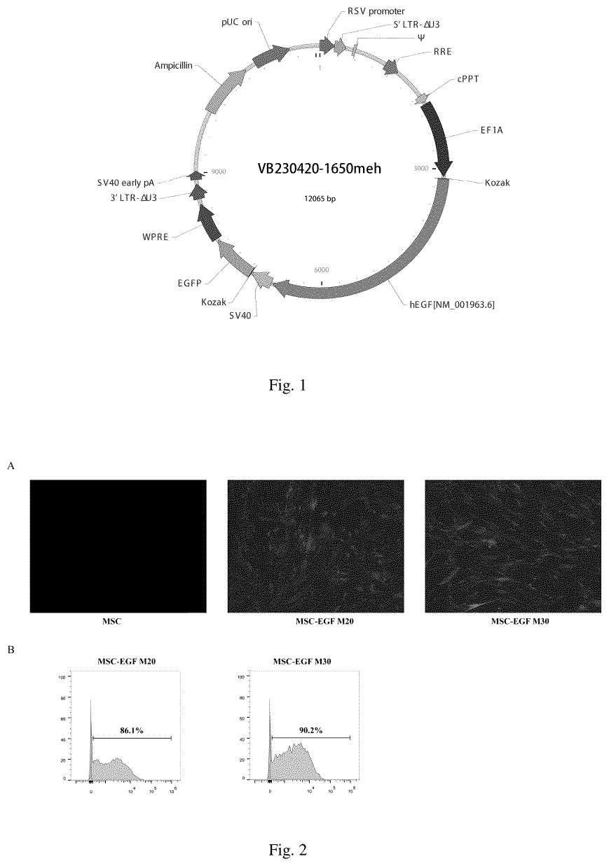

In some embodiments, the disclosure constructs a lentiviral vector overexpressing EGF, and the vector design mapping is shown in FIG. 1. The vector plasmid of the lentiviral vector, sequentially from the 5′LTR region to the 3′LTR region, comprises: an RSV promoter, a 5′LTR-ΔU3, a ψ sequence, an RRE, a cPPT, an EF1α promoter, a Kozak translation initiation sequence, a nucleotide encoding EGF, an EGFP, a WRPE, a 3′LTR-ΔU3, and a SV40 early pA. The vector combines the EF1α promoter sequence and the Kozak translation initiation sequence in upstream of the inserted EGF gene, which ensures high expression of the viral plasmid in stem cells. The constructed lentiviral vector also carries an enhanced GFP sequence (EGFP), which facilitates the observation of transfection efficiency.

The mesenchymal stem cell transfected with the lentiviral vector carrying EGF-coding nucleotides according to the disclosure has significantly increased levels of EGF gene transcription in the transfected cells.

The mesenchymal stem cell transfected with the lentiviral vector carrying EGF-coding nucleotides according to the disclosure has significantly increased levels of EGF protein secretion in the supernatants of the transfected cells.

The mesenchymal stem cell transfected with the lentiviral vectors carrying EGF-coding nucleotides according to the disclosure may be used to prepare pharmaceutical compositions. The pharmaceutical compositions may be used in the treatment of diseases such as wound surface repair and healing, tissue repair, wound healing, or in the prevention of scar hyperplasia.

### Definitions

A lentiviral vector system comprises a packaging plasmid, an envelope plasmid, and a vector plasmid, and can be either a two-plasmid system, a three-plasmid system, or a four-plasmid system. The three-plasmid system is a system in which the cis-acting sequence structures required for packaging, reverse transcription and integration are separated from sequences encoding trans-acting proteins in the lentiviral genome and cloned into three separate plasmids, with all of the auxiliary sequences being removed. The four-plasmid system is obtained by modifying the three-plasmid system. Compared with the three-plasmid system, the first change is that the rev gene is placed on a separate expression plasmid, and the addition of a new plasmid increases the safety of the system. The second change is that the tat gene is removed, and a chimeric 5′LTR fused to a heterologous promoter is added to the vector plasmid to initiate expression of the vector plasmid. Additionally, the three-plasmid system and the four-plasmid system also have a vector in which the target gene sequence can be placed, called a vector plasmid.

Epidermal growth factor (EGF) is a prototypical member of the EGF superfamily of peptide growth factors. The precoding protein is subjected to a proteolytic treatment to generate a 53-amino acid epidermal growth factor peptide, which constitutes a single-chain polypeptide of 53 amino acid residues. EGF is a precursor consisting of 1,217 amino acid residues, and a mature EGF is generated from its precursor by proteolysis. EGF is a potent mitogenic factor that acts through high-affinity binding to the cell surface receptor, epidermal growth factor receptor (EGFR/ErbB).

A ψ sequence is the minimal packaging signal required for the capsidation of lentiviral genomes.

“Stem cell” refers to an undifferentiated cell that is capable of long-term self-renewal or of producing at least one identical copy of the initial cell; differentiating into multiple cells at the single-cell level, and in some cases, only a single specific cell type; and achieving regeneration of tissues in vivo. Stem cells are subdivided into totipotent, sub-totipotent, pluripotent and oligopotent stem cells based on their developmental potential.

Mesenchymal stem cells (MSCs) have been widely used in clinical research of regenerative medicine and autoimmune diseases due to their multidirectional differentiation potential and immunomodulatory functions. Unlike other stem cells, such as hematopoietic stem cells, MSCs are a class of stem cells capable of amplification in vitro. In the disclosure, UMSCs refer to MSCs obtained from umbilical cords, in particular human umbilical cord MSCs (hUC-MSCs). Human umbilical cord MSCs are mesenchymal stem cells originating from the umbilical cord of a newborn baby and have a strong proliferative and multidirectional differentiation capacity. However, it should be understood by those skilled in the art that the source of MSCs is not limited to human umbilical cord MSCs, and that other sources of MSCs can also be used in the disclosure.

### Examples

### Example 1. Preparation of Recombinant Stem Cell

### 1. Plasmid Construction

Firstly, an EGF-overexpressing lentiviral vector was designed and synthesized, and the vector design mapping is shown in FIG. 1. The vector plasmid of the lentiviral vector, sequentially from the 5′LTR region to the 3′LTR region, contains: RSV promoter, 5′LTR-ΔU3, ψ-sequence, RRE, cPPT, EF1α promoter, Kozak translation initiation sequence, nucleotides encoding EGF, EGFP, WRPE, 3′LTR-ΔU3 and SV40 early pA. The vector combined the EF1α promoter sequence and the Kozak translation initiation sequence in upstream of the inserted EGF gene (SEQ ID NO: 1), which ensured the high expression of the viral plasmid in stem cells. The constructed lentiviral vectors also carried an enhanced GFP sequence to facilitate the observation of transfection efficiency. Due to the long EGF sequence, the above elements have reached the maximum carrying capacity of the lentivirus, and it is neither possible to carry the sequences of the drug screening marker antibiotic Puro, which are routinely required to be retained, nor to add additional sequences of the signal peptide, linker region or transmembrane region.

The insertion sequence and restriction endonuclease sites on the vector backbone were analyzed, and the enzyme that could cut the appropriate bands was selected to digest the plasmid DNA. The digested DNA was subjected to agarose gel electrophoresis, and was stained with ethidium bromide EtBr, and the sizes of the DNA fragments were determined, and primers targeting the vector backbone and/or the insertion sequence were designed. The nucleic acid sequence was identified by Sanger sequencing and the alignment was completely correct. The viral vector plasmid was successfully constructed.

### 2. Infection Methods

After the successful construction of the viral vector plasmid, viral packaging was performed, and then hUC-MSCs were transfected. On DO of transduction, hUC-MSCs of the P4 to P8 generation were selected, and the cells were seeded into 6-well plates at a density of 1×10⁵ to 2×10⁵/well, with the culture medium of DMEM/F12+10% FBS, and cultured in an incubator at 37° C. 5% CO₂ for 18 to 20 h. The transfection was performed after the cell density reached 30% to 50% of confluence. On the day of transduction (D1), the viral solution was thawed on ice and mixed gently. The count of viruses was aspirated according to the MOI (MOI was about 20 and 30 respectively), added to the culture medium and mixed gently. It was suitable that the amount of culture medium covered the surface area of the culture medium and the amount was 100 μL/mL, and the amount of culture medium used in a 6-well plate was 1 mL/well. The original culture medium was aspirated out and the culture medium added with viruses was added to the 6-well plate on which hUC-MSCs were cultured. At the same time, 5 to 8 μg/mL of an auxiliary transfection reagent, polybrene, was added to each well, and mixed well so that the virus covered every cell, and the recombinant stem cells MSC-EGF M20 and MSC-EGF M30 were prepared by culturing in the incubator at 37° C. 5% CO₂ for 6 to 8 h. Excessive exposure to polybrene can cause cell toxicity, so the transduction time should not be too long, otherwise the cell status may be affected. Meanwhile, the virus solution with empty vector was used as a blank control for transduction in the same way, and wild-type MSCs of the blank control group were prepared.

### Example 2. Identification of Infection Efficiency

On the second day after transduction, the culture medium containing viruses was aspirated, and fresh DMEM/F12+10% FBS culture medium was added and cultured overnight in the incubator at 37° C. 5% CO₂. Usually, the genes carried by lentivirus began to be expressed on the second day of transduction, and green fluorescence can be observed 48 to 72 h after transfection. Fluorescent expression was observed daily, and strong expression of green fluorescent protein of GFP was observed by confocal microscopy after 72 h (FIG. 2A). The percentage of green fluorescence was counted, while the efficiency of infection was confirmed by flow cytometry. The results showed that the effective infection rate was exceed 86% at an MOI of 20, and 90.2% at an MOI of 30 (FIG. 2B).

### Example 3. Identification of Protein Expression

RNA was extracted from wild-type mesenchymal stem cells (MSCs) of the blank control group and lentivirus-infected mesenchymal stem cells containing integrated EGF sequences (EGF MSCs) prepared in Example 1, and the RNA was subjected to reverse transcription experiments to obtain the cDNA using the PrimeScript RT-PCR Kit (TaKaRa). Then, the method of qPCR was used to detect the changes of EGF at the mRNA level, with GAPDH as an internal reference.

The sequences of PCR primers for fluorescence quantitative PCR detection of the internal reference GAPDH were as follows:

Forward primer F:
(SEQ ID NO: 2)
5′-CGAGATCCCTCCAAAATCAA-3′
and
Reverse primer R:
(SEQ ID NO: 3)
5′-ATCCACAGTCTTCTGGGTGG-3′.

The sequences of PCR primers for fluorescent quantitative PCR detection of EGF were as follows:

Forward primer F:
(SEQ ID NO: 4)
5′-TCTTGCTGTGGACTGGATTG-3′;
and
Reverse primer R:
(SEQ ID NO: 5)
5′-AGCAATTCCTCGTGGTTGAG-3′.

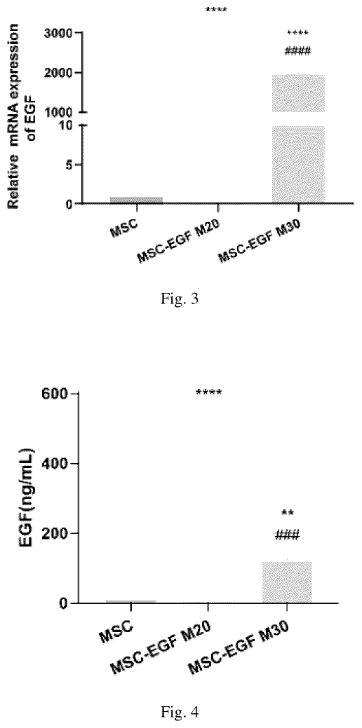

As shown in FIG. 3, the experimental results showed that compared with wild-type mesenchymal cells (MSCs), EGF in lentivirus-infected recombinant mesenchymal stem cells (MSC-EGF) containing integrated EGF sequences was increased at the mRNA level, which significantly increased the expression of EGF. Specifically, the transcription of the EGF gene was increased by about 2,000 times at an MOI of 30, and by 2,500 times at an MOI of 20.

### Example 4. Identification of Protein Secretion Levels in Cell Supernatants

To confirm the increased secretion of EGF secreted by MSCs, an enzyme-linked immunosorbent assay (ELISA) was used for analysis.

Wild-type mesenchymal cells (MSCs) from the blank control group and mesenchymal stem cells (MSC-EGF) infected by the lentivirus integrated with EGF sequence prepared in Example 1 were cultured in a dish, respectively. When the cell confluence reached 80% to 90%, the cell culture plate or cell suspension was taken, and the cell culture plate was washed twice with PBS, and the cell was disrupted by freezing and thawing so that the intracellular components were released, followed by centrifuging at 4° C., 2500 rpm for 15 min, and the supernatant was obtained. 100 μL of culture supernatant and different concentrations of standards were added to a plate coated with antibodies, with three wells per group. The plate was sealed with a sealing tape, and incubated at 37° C. for 90 min and then was washed with 1×washing buffer for 5 times. The plate was added with a biotinylated antibody working solution at 50 μL/well, covered with sealing film, and incubated at 37° C. for 90 min. The plate was washed with 1×washing buffer for 5 times. The plate was added with a horseradish peroxidase-conjugated streptavidin-HRP (streptavidin-HRP) working solution at 100 μL/well, covered with sealing film, and incubated at 37° C. for 30 min. The plate was washed 5 times. The plate was added with TMB chromogenic solution at 100 μL/well, and incubated at 37° C. for 15 min. The reaction was terminated by adding with termination solution at 100 μL/well. The concentration was calculated by reading the values at a detection wavelength of 450 nm.

As shown in FIG. 4, the results indicated that the recombinant mesenchymal stem cells infected with EGF lentivirus (MSC-EGF) were able to secrete more EGF compared with wild-type mesenchymal cells (MSCs). The expression level of EGF in the control group was low, but the protein secretion level in the supernatants of the overexpressed recombinant stem cells was significantly increased. The protein secretion increased from undetectable level (below 10 pg/mL) prior to infection to about 100 ng/ml (at an MOI of 20), and the secretion level increased by nearly 10,000 times. In particular, at an MOI of 20, the protein secretion was increased to about 450 ng/mL, and the secretion level increased by nearly 40,000 times.

Some exemplary embodiments of the disclosure have been illustrated and described, however, the disclosure is not limited to the disclosed embodiments. Rather, those skilled in the art will recognize that a number of modifications and changes may be made to the described embodiments without departing from the spirit and scope of the disclosure as described in the claims.

The technical solutions of the disclosure are not limited to the above specific embodiments, and any technical modifications made based on the technical solutions of the disclosure all fall within the scope of the disclosure.

What is claimed is:

1. A lentiviral vector comprising a vector plasmid, wherein the vector plasmid comprises a 5′LTR containing a ψ sequence, a 3′LTR, a target gene sequence between the 5′LTR and the 3′LTR, and a promoter sequence and a translation initiation sequence operably linked to the target gene sequence, and the target gene sequence is a nucleotide sequence encoding epidermal growth factor (EGF).

2. The lentiviral vector of claim 1, wherein the nucleotide sequence encoding EGF is a nucleotide sequence shown in SEQ ID NO: 1 or a nucleotide sequence having at least 85%, at least 90% or at least 95% sequence identity to SEQ ID NO: 1.

3. The lentiviral vector of claim 1, wherein the promoter of the 5′LTR is selected from the group consisting of a cytomegalovirus CMV promoter, a Rous sarcoma virus RSV promoter, and a simian virus SV40 promoter; and/or
the promoter operably linked to the nucleotide encoding EGF is selected from the group consisting of a short elongation factor 1A (EF1α) promoter or a transcriptionally active fragment thereof, an RSV promoter, and a simian virus SV40 promoter.

4. The lentiviral vector of claim 3, wherein the promoter operably linked to the nucleotide encoding EGF is a short elongation factor 1A (EF1α) promoter.

5. The lentiviral vector of claim 3, wherein the promoter of the 5′LTR is a Rous sarcoma virus RSV promoter.

6. The lentiviral vector of claim 3, wherein the nucleotide encoding EGF is operably linked to an EF1α promoter and a Kozak translation initiation sequence.

7. The lentiviral vector of claim 1, wherein the vector plasmid further comprises a nucleotide encoding a screening marker, a woodchuck hepatitis virus post-transcriptional regulatory element WPRE, a retroviral export element, and a central polypurine region (cPPT) or a central termination sequence (CTS).

8. The lentiviral vector of claim 7, wherein the screening marker is selected from one or more of Luciferase, fluorescent protein, streptavidin binding peptide, puromycin resistance marker, ampicillin resistance marker, kanamycin resistance marker, and neomycin resistance marker.

9. The lentiviral vector of claim 7, wherein the screening marker is an enhanced green fluorescent protein (EGFP).

10. The lentiviral vector of claim 7, wherein the retroviral export element is selected from the group consisting of a human immunodeficiency virus rev response element RRE and a hepatitis B virus post-transcriptional regulatory element HPRE.

11. The lentiviral vector of claim 7, wherein the cPPT is a cPPT of HIV1.

12. The lentiviral vector of claim 7, wherein the CTS is a CTS of HIV1.

13. The lentiviral vector of claim 1, wherein the vector plasmid comprises sequentially from the 5′LTR region to the 3′LTR region: an RSV promoter, a 5′LTR-ΔU3, a ψ sequence, an RRE, a cPPT, an EF1α promoter, a Kozak translation initiation sequence, a nucleotide encoding EGF, an EGFP, a WRPE, a 3′LTR-ΔU3, and an SV40 early pA.

14. A recombinant stem cell, wherein the recombinant stem cell comprises the lentiviral vector of claim 1.

15. The recombinant stem cell of claim 14, wherein the recombinant stem cell is a mesenchymal stem cell.

16. The recombinant stem cell of claim 15, wherein the mesenchymal stem cell is a human umbilical cord mesenchymal stem cell.

17. A method for the preparation of the recombinant stem cell of claim 14, wherein the method comprises the following steps:
(1) constructing the lentiviral vector;
(2) transfecting a stem cell with the lentiviral vector constructed in step (1) to obtain the recombinant stem cell; and
(3) culturing the recombinant stem cell obtained in step (2).

18. A pharmaceutical composition, wherein the pharmaceutical composition comprises the lentiviral vector of claim 1 or a recombinant stem cell comprising the lentiviral vector, and a pharmaceutically acceptable excipient or carrier.

19. A method for the treatment of wound surfaces repair and healing, tissue repair, or wound healing, or for the prevention of scar hyperplasia in a subject in need thereof, comprising administrating to the subject a therapeutically effective amount of the lentiviral vector of claim 1, a recombinant stem cell comprising the lentiviral vector, or a pharmaceutical composition that comprises the lentiviral vector or a recombinant stem cell comprising the lentiviral vector.

20. The method of claim 19, wherein the wound surfaces include wound surfaces caused by corneal injury, burns, scalds, surgeries, or chronic ulcers.
